(19) United States
(12) Patent Application Publication
(10) Pub. No.: US 2026/0137651 A1
(43) Pub. Date: May 21, 2026

(54) MULTI-COMPARTMENT NANOEMULSION FOR INTRANASAL DRUG DELIVERY

(71) Applicant: KING ABDULAZIZ UNIVERSITY, Jeddah (SA)

(72) Inventors: Haythum Osama Tayeb, Jeddah (SA); Hossam H. Tayeb, Jeddah (SA); Jawza Khaled Alumutairi, Jeddah (SA); Hussam Aly Sayed Murad, Jeddah (SA); Raed Felimban, Jeddah (SA); Badrah Saeed Alghamdi, Jeddah (SA)

(73) Assignee: KING ABDULAZIZ UNIVERSITY, Jeddah (SA)

(21) Appl. No.: 18/950,287

(22) Filed: Nov. 18, 2024

(51) Int. Cl. A61K 31/4015; A61K 9/00; A61K 31/19; A61K 47/26; A61K 47/44

(52) U.S. Cl. CPC A61K 31/4015; A61K 9/0043; A61K 31/19; A61K 47/26; A61K 47/44

(57) ABSTRACT

A method of intranasal drug delivery including administering a multi-compartment nanoemulsion to a nasal cavity of a subject in need thereof, where the multi-compartment nanoemulsion includes a water-in-oil core emulsion having an oil phase encompassing a first aqueous phase, the oil phase comprising a first surfactant and a fatty acid and the first aqueous phase comprising levetiracetam and valproic acid dissolved therein. The multi-compartment nanoemulsion further includes a second aqueous phase encompassing the water-in-oil core emulsion, the second aqueous phase comprising a second surfactant and a co-surfactant in a ratio of the second surfactant to the co-surfactant of 1:4 to 4:1. The multi-compartment nanoemulsion has an average droplet size of less than 100 nm and a polydispersity index (PDI) of 0.1 to 0.2.

### STATEMENT OF ACKNOWLEDGEMENT

This research work was funded by Institutional Fund Projects under grant no (IFPRC-171-140-2020). Applicants acknowledge technical and financial support provided by King Abdulaziz University, the Ministry of Education, Jeddah, Saudi Arabia.

### BACKGROUND

### Technical Field

The present disclosure is directed to a multi-compartment water-in-oil-in-water nanoemulsion (W/O/W-NE) for intranasal drug delivery.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Intranasal drug delivery is a non-invasive and effective route for the administration of drugs to the brain at pharmacologically relevant concentrations. Intranasal drug delivery allows drugs which do not cross the blood brain barrier (BBB) to enter the central nervous system (CNS), thus eliminating the need for systemic drug delivery and reducing unwanted systemic side effects. The CNS includes the brain, the brain stem, and the spinal cord. The CNS is isolated from the rest of the body by several membranes that both cushion and safeguard the brain, the brain stem, and the spinal cord. For example, the membranes that form the blood-brain barrier (BBB) protect the brain from certain contents of the blood. The blood-cerebrospinal fluid barrier (BCSFB) safeguard other portions of the CNS from many chemicals and microbes.

Targeting of drugs to the central nervous system (CNS) is a challenging task. Typically, only small lipophilic molecules may cross the BBB via transcellular passive diffusion, although some peptides and peptide analogs are known to cross the BBB. Additionally, some low molecular weight drugs may cross the BBB, however, high molecular weight hydrophilic substances are generally blocked from crossing the BBB under normal conditions. Invasive methods may be employed to transport drugs across the BBB by temporarily disrupting the integrity of the BBB integrity, such as osmotic shock to the endothelial layer by administering mannitol or by ultrasound disruption. The disadvantages associated with these invasive procedures are increased cost, long-term hospitalization, and possible neuron damage by cytotoxic molecules crossing the BBB.

Epilepsy is a disorder of the brain characterized by chronic, recurring seizures. Seizures occur due to uncontrolled discharges of electrical activity in the brain. Symptoms of seizure include sudden, involuntary, disruptive, and often destructive sensory, motor, and cognitive phenomenon. It is usually associated with physical harm to the body (e.g., tongue biting, limb breakage, and burns), a total loss of consciousness, and incontinence. A seizure usually starts with spontaneous shaking of an arm or leg and progresses to rhythmic movement of the entire body, loss of consciousness, and voiding of urine or stool.

The approaches to drug therapy of epilepsy are directed at the control of symptoms by chronic administration of antiepileptic/anticonvulsant drugs (AEDs). These AEDs are administered orally or intravenously, but alternative routes of administration are increasingly important to overcome negative side effects of systemic drug delivery methods, as described before. Intranasal administration is a potentially attractive route for AEDs because it is possible to reach the brain without needing to cross the blood brain barrier. Further, intranasal administration allows the drug to avoid first-pass metabolism. Intranasal administration may also provide an easy and non-invasive route of drug administration, leading to increases in patient compliance. However, there are some limitations with intranasal administration including, but not limited to, small dosage size due to the small size of the nasal cavity, low drug bioavailability due to the loss of drug through swallowing, inhalation, and other clearance mechanisms, nasal cavity environment has a small surface area and may comprise enzymes that break down certain drugs thus limiting how much of the drug is absorbed, drugs with a high molecular weight may have trouble crossing the nasal mucosa. Further, the intranasal drug also needs excellent aqueous solubility or must be able to be formulated using solubilizing agents.

Accordingly, it is one object of the present disclosure to develop a intranasal drug delivery system that can facilitate delivery of water-soluble anti-seizure drugs, bypass the BBB, and achieve a desirable drug bioavailability inside the body.

### SUMMARY

In an exemplary embodiment, a method of intranasal drug delivery is described. The method comprises administering a multi-compartment nanoemulsion to a nasal cavity of a subject in need thereof. The multicompartment nanoemulsion comprises a water-in-oil core emulsion having an oil phase encompassing a first aqueous phase, the oil phase comprising a first surfactant and a fatty acid and the first aqueous phase comprising levetiracetam and valproic acid dissolved therein. The multi-compartment nanoemulsion further comprises a second aqueous phase encompassing the water-in-oil core emulsion, the second aqueous phase comprising a second surfactant and a co-surfactant in a ratio of the second surfactant to the co-surfactant of 1:4 to 4:1. The water-in-oil core emulsion has an average droplet size of 20 d·nm or less and the multi-compartment nanoemulsion has an average droplet size of less than 100 d·nm. The first surfactant is polyglyceryl-3 polyricinoleate and the co-surfactant is a polysorbate.

The multi-compartment nanoemulsion has an average droplet size of less than 100 nanometers (d·nm). The multi-compartment nanoemulsion has a polydispersity index (PDI) of 0.1 to 0.2.

In some embodiments, the method further comprises preparing the first aqueous phase by mixing levetiracetam and valproic acid in water. The method further comprises forming the oil phase, then dispersing the first aqueous phase into the oil phase to obtain the water-in-oil core emulsion. The method further comprises dispersing the water-in-oil core emulsion into the second aqueous phase, then sonicating to obtain the multi-compartment nanoemulsion.

In some embodiments, the second surfactant is present in an amount of 1 to 10 wt. %.

In some embodiments, the co-surfactant is present in an amount of 1 to 10 wt. %.

In some embodiments, the multi-compartment nanoemulsion has an average droplet size of less than 85 d·nm.

In some embodiments, the fatty acid is at least one selected from the group consisting of a coconut oil, a palm kernel oil, a caproic acid, a caprylic acid, a capric acid, and a lauric acid.

In some embodiments, the second surfactant is present in an amount of 7 wt. %.

In some embodiments, the co-surfactant is present in an amount of 7 wt. %.

In some embodiments, the second surfactant is at least one selected from the group consisting of a glyceryl citrate, a glyceryl lactate, a glyceryl linoleate, and a glyceryl oleate.

In some embodiments, the sonicating comprises sonicating the aqueous suspension at 25 to 65% amplitude bursts for 15 to 60 seconds (see); and holding the aqueous suspension in an ice bath between each burst for 0.5 to 6 minutes (min).

In some embodiments, the forming of the oil phase comprises adding the fatty acid to the first aqueous phase, then adding the first surfactant to the fatty acid in a ratio of first surfactant to fatty acid of 1:1 to 3:1.

In some embodiments, the sonicating comprises sonicating the aqueous suspension at 45% amplitude bursts for 45 sec; and holding the aqueous suspension in an ice bath between each burst for 1 min.

In some embodiments, the multi-compartment nanoemulsion has a PDI of 0.158.

In some embodiments, the multi-compartment nanoemulsion has an average droplet size of 77.7±0.55 d·nm.

In some embodiments, the water-in-oil core emulsion has a PDI of 0.03 to 0.06.

In some embodiments, the water-in-oil core emulsion has a PDI of 0.058.

In some embodiments, the water-in-oil core emulsion has an average droplet size of 18.5±0.44 d·nm.

In some embodiments, the fatty acid is coconut oil.

In some embodiments, the levetiracetam is present in the first aqueous phase in a concentration of 100 to 150 mg/mL.

In some embodiments, the valproic acid is present in the first aqueous phase in a concentration of 30 to 70 mg/mL.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic diagram of a method of intranasal drug delivery, according to certain embodiments.

FIG. 2 is a schematic diagram depicting the structure of the water-in-oil core nanoemulsion (W/O-NE) and the water-in-oil-in-water multi-compartment nanoemulsion (W/O/W-NE), according to certain embodiments.

FIG. 3 is a graph depicting average droplet size of W/O-NE and W/O/W-NE in diameter of nanometers (d·nm), according to certain embodiments.

FIG. 4 is a graph depicting impact of varying storage temperatures on the size of W/O/W-NEs, according to certain embodiments.

FIG. 5 is a graph depicting impact of phosphate buffered saline (PBS) solution on the size of W/O/W-NEs, according to certain embodiments.

FIG. 6 is a schematic diagram of nanoemulsion preparation process, according to certain embodiments.

FIG. 7A to FIG. 7C illustrate photomicrographs of sheep nasal mucosa treated with (A) PBS at pH of 6.5, (B) isopropyl alcohol, and (C) a solution comprising the W/O/W-NEs, according to certain embodiments.

FIG. 8 is a diagram of the experimental procedure and treatment schedule of the W/O/W-NEs in an in vivo study, according to certain embodiments.

FIG. 9A to FIG. 9D depict the effect of delivery of a combination of valproic acid (VPA) and levetiracetam (LEV), administered as an intranasal W/O/W-NEs, in pentylenetetrazol (PTZ) induced seizures on (A) latency to seizure, (B) number of generalized tonic-clonic seizures (GTCS), (C) duration of GTCS, and (D) Racine score.

FIG. 10A to FIG. 10D depicts the concentrations (mcg) of LEV and VPA in the serum (10A and 10B) and the brain (10C and 10D) 60 minutes after administration, as measured with Liquid Chromatography-Mass Spectrometry (LC-MS)

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Embodiments of the present disclosure will now be described more fully hereinafter with reference to the accompanying drawings, in which some, but not all embodiments of the disclosure are shown.

As used herein, the words “about,” “approximately,” or “substantially similar” may be used when describing magnitude and or position to indicate that the value and/or position described is within a reasonable expected range of values and/or positions. For example, a numeric value may have a value that is +/−0.1% of the stated value (or range of values), +/−1% of the slated value (or range of values), +/−2% of the stated value (or range of values), +/−5% of the slated value (or range of values), +/−10% of the staled value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

As used herein, “nanoemulsion” refers nano-sized emulsions, which are manufactured for improving the delivery of active pharmaceutical ingredients. These are thermodynamically stable isotropic systems in which two immiscible liquids are mixed to form a single phase by means of an emulsifying agent.

As used herein, “emulsifying agent” also known as an emulsifier, refers to a substance that mixes two immiscible liquids, like oil and water, to create a uniform dispersion called an emulsion. Emulsifiers are surface-active ingredients that work by forming a protective barrier around dispersed droplets, decreasing interfacial tension between the oil and water phases to precent droplets from forming and aggregating, and charging the surface of the drop which reduces the physical contact between the droplets. Emulsifying agents may also be referred to as surface-active agents and surfactants.

As used herein, “co-surfactant” refers to a substance that is used in combination with a surfactant to improve the surfactant's performance. Co-surfactants work by aiding in the formation of stable microemulsions, increasing the solubility of drugs by reducing the interfacial tension between the drug and the surfactant, and aiding in the formation of nanoparticles. Co-surfactants are generally amphiphilic molecules that accumulate at the interfacial layer with the surfactant. Suitable co-surfactants include, but are not limited to, alcohols and non-ionic surfactants.

As used herein “multi-compartment nanoemulsion” refers to a water-in-oil-in-water nanoemulsion or system where an oil phase encompasses an first aqueous phase, the oil phase being further encompassed by a second aqueous phase.

As used herein, the term “intranasal(ly)” refers to administration of the compositions of the present invention to the surface of the skin and mucosal cells/tissues of the nasal passages, e.g., nasal mucosa, sinus cavity, nasal turbinates, or other tissues and cells which line the nasal passages.

A used herein, “pharmaceutically acceptable” or “pharmacologically acceptable,” as used herein, refer to compositions that do not substantially produce adverse allergic or adverse immunological reactions when administered to a host (e.g., an animal or a human). Such formulations include any pharmaceutically acceptable dosage form. Examples of such pharmaceutically acceptable dosage forms include, but are not limited to, dips, sprays, seed dressings, stem injections, lyophilized dosage forms, sprays, and mists. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

As used herein the term “subject” refers to a warm-blooded animal, and preferably mammals, such as, for example, cats, dogs, horses, cows, pigs, mice, rats and primates, mammals including humans. The preferred mammal is a human. In various embodiments, the subject can be a human (e.g., adult male, adult female, adolescent male, adolescent female etc.) under the care of a physician or other healthcare worker in a hospital, psychiatric care facility, as an outpatient, or other clinical context. In certain embodiments the subject may not be under the care or prescription of a physician or other healthcare worker.

As used herein the term “nasal cavity” refers to the space inside a subject's nose that allows air to pass through during breathing. The nasal cavity the first part of the respiratory system and is located above the roof of the mouth, in humans, curving down to connect with the throat. The nasal cavity of humans has a nasal septum dividing the cavity into two sections, also known as nasal passages or fossae. The nasal cavity of a human has a roof, floor, medial wall, and lateral wall. The lateral walls are made up of the nasal conchae, which are shell-shaped structures. Each nasal passage of a human has three regions known as the nasal vestibule, respiratory region, and olfactory region. The nasal cavity is lined with a mucous membrane referred to as nasal mucosa. The nasal mucosa offers numerous benefits as a target issue for drug delivery, such as a large surface area for delivery, rapid drug onset, potential for CNS delivery, and no first-pass metabolism

As used herein, “particle size” and “average particle size” are used synonymously with the average diameter of the particles in a given liquid, e.g., droplets diameter or micelle diameter in an emulsion.

As used herein “anticonvulsant” and “antiepileptic” both refer to a type of drug that is used to prevent or treat seizures or convulsions by controlling abnormal electrical activity in the brain.

As used herein “valproic acid” or “valproate” (VPA) refers to a branched chain dicarboxylic acid having broad anticonvulsant activity. VPA is effective in the prevention/treatment of absence seizures, myoclonic seizures, partial seizures, and tonic-clonic seizures.

As used herein “levetiracetam” (LEV) refers to a pyrrolidinone having broad anticonvulsant activity. LEV is effective in the prevention/treatment of partial seizures, myoclonic seizures, and tonic-clonic seizures. LEV may also be useful as an adjunctive therapeutic for focal seizures, myoclonic seizures, and primary generalized seizures.

As used herein, “polydispersity index” (PDI), refers to the measure of the distribution of the particle sizes within a given sample. The numerical value of PDI ranges from 0.0 (for a perfectly uniform sample with respect to the particle size) to 1.0 (for a highly polydisperse sample with multiple particle size populations). PDI values may vary due to the particle size distribution of a sample or the presence of agglomerates or aggregates in the sample. PDI is measured using dynamic light scattering (DLS). Successful formulation of safe, stable, and efficient nanoemulsions for intranasal delivery require the nanoemulsion to have a substantially monodisperse population of nanoparticles having a certain droplet size. The particle size distribution may be controlled by the selection of the surfactants and co-surfactants utilized during preparation of the nanoemulsion. PDI may be measured according to International Standards Organization (ISO) 22,412:2017. The ISO has established that PDI values <0.05 are more common to monodisperse samples, while PDI values >0.7 more common to broad size (i.e., polydisperse) distribution of particles.

Aspects of the present disclosure are directed toward a multi-compartment nanoemulsion (W/O/W-NE) as an intranasal drug for in-vivo applications. This multi-compartment nanoemulsion shows the promising capability to simultaneously encapsulate levetiracetam and valproic acid in an aqueous phase. This formulation is considerable progress for delivering medications directly and rapidly to the brain via olfactory neurons, offering a safe and efficient alternative to oral and intravenous administration of AEDs in treating epilepsy or epileptic seizures.

Epilepsy is a cerebral disorder characterized by recurrent seizures. Types of epilepsy may include, but are not limited to, generalized epilepsy, for example, childhood abscess epilepsy, juvenile myoclonic epilepsy, epilepsy with generalized epileptic seizures on waking, West syndrome, Lennox-Gastaut syndrome, and focal epilepsy, for example, frontal lobe epilepsy, e.g. childhood epilepsy.

The epileptic seizures described in this application may include seizures, acute recurrent seizures, cluster epileptic seizures, continuous epileptic seizures, persistent epileptic seizures, prolonged epileptic seizures, recurrent seizures, epileptic seizures of status epilepticus, for example, refractory convulsive status epilepticus, epileptic seizures of non-convulsive status epilepticus, refractory epileptic seizures, myoclonic epileptic seizures, tonic epileptic seizures, tonic-clonic epileptic seizures, simple partial epileptic seizures, complex partial epileptic seizures, secondary generalized epileptic seizures, atypical absence epileptic seizures, absence epileptic seizures, atonic epileptic seizures, mild rolandic epileptic seizures, fibril epileptic seizures, affective epileptic seizures, focal epileptic seizures, gelastic epileptic seizures, epileptic seizures of generalized onset, infant cramps, Jacksonian epileptic seizures, massive bilateral myoclonic epileptic seizures, multifocal epileptic seizures, neonatal epileptic seizures, night epileptic seizures, occipital lobe epileptic seizures, post-traumatic epileptic seizures, mild epileptic seizures, mild childhood epileptic seizures, visual reflex epileptic seizures, and abstinence seizures.

According to a first aspect of the present disclosure, a multi-component nanoemulsion is described. In one embodiment, the nanoemulsion comprises a first aqueous phase with levetiracetam and valproic acid dissolved therein. Surrounding the first aqueous phase is an oil phase. The oil phase comprises a first surfactant and a fatty acid, which aid in stabilizing the emulsion by increasing the interaction between various layers in the system. The multi-compartment nanoemulsion further comprises a second aqueous phase which encompasses the oil phase. The second aqueous phase comprises a second surfactant and a co-surfactant. The ratio of the second surfactant to the co-surfactant is in a range of 1:4 to 4:1. The second surfactant lowers the surface tension between the oil phase and the second aqueous phase, while the co-surfactant enhances emulsification.

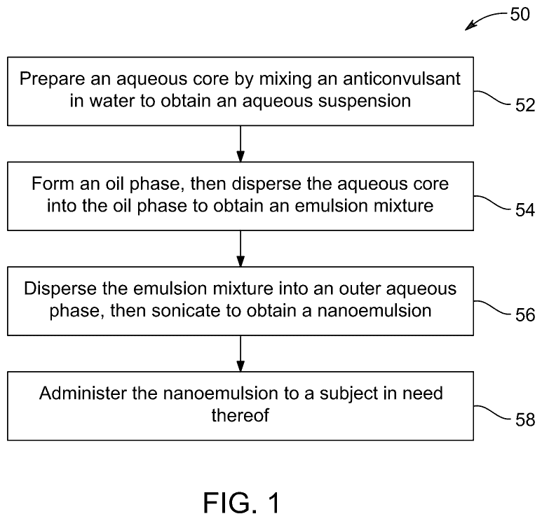

According to another aspect of the present disclosure, a method for intranasal administration of the nanoemulsion is described (FIG. 1). The order in which the method 50 is described is not intended to be construed as a limitation, and any number of the described method steps can be combined to implement the method 50. Additionally, individual steps may be removed or skipped from the method 50 without departing from the spirit and scope of the present disclosure.

At step 52, the method 50 comprises preparing the first aqueous phase by mixing the levetiracetam and valproic acid in water to obtain an aqueous suspension. In some embodiments, other AEDs other than levetiracetam and valproic acid may be used in the first aqueous phase. The AED is preferably water-soluble, however, in some embodiments, AEDs that are not water soluble can be converted to their water-soluble salts and then prepared in the aqueous suspension. Suitable examples of water-soluble AEDs include, but are not limited to, gabapentin, levetiracetam, valproic acid, topiramate, ethosuximide, zonisamide, to name a few.

In some embodiments, poorly water-soluble AEDs may be used as well. For example, poorly water-soluble AEDs include 5,5-diphenylhydantoin, benzodiazepine, carbamazepine, clonazepam, clorazepate, diazepam, divalproex, ethosuximide, felbamate, fosphenytoin, gabapentin, lamotrigine, methsuximide oxcarbazepine, phenytoin, pregabalin, tiagabine, or their pharmaceutically acceptable salt or prodrug. However, these AEDs need to be converted to their water-soluble forms or may require the use of aqueous solvents, other than water. Certain examples of such aqueous solvents include aqueous salines, aqueous surfactant solutions (e.g., water mixed with surfactants such as polysorbates or lecithin to overcome solubility or stability issues), aqueous co-solvent systems (e.g., water mixed with a secondary solvent, such as ethanol, propylene glycol, or glycerin), aqueous electrolyte solutions (e.g., water comprising dissolved electrolytes such as NaCl or KCl), buffered aqueous solutions (e.g., water comprising buffers such as phosphate buffered saline or citrate buffers), and other like aqueous solvents. The choice of the aqueous solvent may be determined by any selection method known in the art. In a preferred embodiment, the aqueous suspension comprises water as the aqueous solvent.

In one embodiment, the aqueous solvent comprises at least 60% or more water, preferably 65% or more, preferably 70% or more, preferably 75% or more, preferably greater 80% or more, preferably 85% or more, preferably 90% or more, preferably 92% or more, preferably 94% or more, preferably 95% or more, preferably 97% or more, preferably 98% or more, preferably 99% or more, and most preferably 100% water. In a preferred embodiment, the first aqueous phase is made of 100% water to solubilize water-soluble AEDs, thereby acting as a vehicle for drug delivery.

In some embodiments, the solubility of the levetiracetam and valproic acid in water can be enhanced by mixing via stirring, swirling, agitation, shaking, or the like. In other embodiments, the solubility can be enhanced by adjusting the pH using certain buffering agents. In a preferred embodiment, the pH of the aqueous solution is between 6 to 8, preferably about 7.

In one embodiment, the levetiracetam is present in the first aqueous phase in a concentration of 100 to 150 mg/mL, preferably 105 to 145 mg/mL, preferably 110 to 140 mg/mL, preferably 115 to 135 mg/mL, preferably 120 to 135 mg/mL, preferably 125 to 135 mg/mL, preferably 130 to 135 mg/mL, most preferably 133 mg/mL. In one embodiment, the valproic acid is present in the first aqueous phase in a concentration of 30 to 70 mg/mL, preferably 35 to 65 mg/mL, preferably 40 to 60 mg/mL, preferably 45 to 55 mg/mL, most preferably 55 mg/mL.

At step 54, the method 50 comprises forming the oil phase, and then dispersing the first aqueous phase into the oil phase to obtain the water-in-oil core emulsion. The oil phase comprises a first surfactant and a fatty acid. The fatty acid may be one selected from a coconut oil, a palm kernel oil, a caproic acid, a caprylic acid, a capric acid, and a lauric acid. In a preferred embodiment, the fatty acid is coconut oil. Coconut oils may comprise several fatty acids such as lauric acid, myristic acid, palmitic acid, caprylic acid, capric acid, oleic acid, linoleic acid, stearic acid, octanoic acid, decanoic acids, and the like. In some embodiments, animal oil, plant oil, vegetable oil, natural oil, synthetic oil, hydrocarbon oils, silicone oils, semi-synthetic derivatives thereof, and combinations thereof, may also be used in alone or in combination with the coconut oil. The weight percentage (wt. %) of fatty acid is 10 to 50 wt. %, preferably 12 to 48 wt. %, preferably 14 to 46 wt. %, preferably 16 to 44 wt. %, preferably 18 to 42 wt. %, preferably 20 to 40 wt. %, preferably 22 to 40 wt. %, preferably 24 to 40 wt. %, preferably 26 to 40 wt. %, preferably 28 to 40 wt. %, preferably 30 to 40 wt. %, preferably 32 to 40 wt. %, preferably 34 to 40 wt. %, preferably 36 to 40 wt. %, preferably 38 to 40 wt. %, most preferably about 40 wt. % in the oil phase.

In some embodiments, the first surfactant may be, but is not limited to, sorbitan, glycerol esters, polyethylene glycol esters, block polymers, acrylic polymers, ethoxylated fatty esters, ethoxylated alcohols, ethoxylated fatty acids, monoglycerides, silicon-based surfactants, and polysorbates. In one embodiment, the first surfactant is at least one selected from a polyglyceryl-4 cocoate, a polyglyceryl-3 caprate, a diisostearyl polyglyceryl-3 dilinoleate, a polyglyceryl-3 diisostearate, a polyglyceryl-3 oleate, and a polyglyceryl-3 polyricinoleate. In a preferred embodiment, the first surfactant is polyglyceryl-3 polyricinoleate.

The oil phase is formed by adding the fatty acid to the first aqueous phase, then adding the first surfactant to the fatty acid in a ratio of fatty acid to first surfactant of 1:1 to 3:1. In some embodiments, the first surfactant is added to the fatty acid in a ratio of fatty acid to first surfactant of 1.10:1 to 5:1, preferably 1.20:1 to 4:1, preferably 1.30:1 to 3:1, preferably 1.30:1 to 2:1, most preferably 1.33:1. The first aqueous phase is then dispersed into the oil phase to form the water-in-oil core emulsion. In some embodiments, the water-in-oil core emulsion is subjected to sonication or other modes of agitation using mixers or homogenizers to break the first aqueous phase into tiny droplets and disperse them through the oil. The surfactants play a key role in stabilizing the water-in-oil core emulsion by reducing the surface tension between the oil and the water (present in the first aqueous phase). In one embodiment, the water-in-oil core emulsion is subjected to sonication at a power of 100 to 140 Watts (W) and a frequency of 10 to 30 kHz. In one embodiment, the water-in-oil core emulsion is subjected to sonication at a power of 105 to 135 W, preferably 110 to 130 W, preferably 115 to 125 W, preferably 120 to 125 W, most preferably 125 W. In one embodiment, the water-in-oil core emulsion is subjected to sonication at a frequency of 12 to 28 kHz, preferably 14 to 26 kHz, preferably 16 to 24 kHz, preferably 18 to 22 kHz, most preferably 20 kHz. In a preferred embodiment, the water-in-oil core emulsion is subjected to sonication at a power of 125 W and a frequency of 20 kHz for 15 to 60 sec at 20 to 65% amplitude bursts for 1 to 20 bursts. In one embodiment, the water-in-oil core emulsion is subjected to sonication for 20 to 55 sec, preferably 25 to 50 sec, preferably 30 to 45 sec, preferably 35 to 45 sec, preferably 40 to 45 sec, most preferably 45 sec. In one embodiment, the water-in-oil core emulsion is subjected to sonication at 25 to 60% amplitude bursts, preferably 30 to 55% amplitude bursts, preferably 35 to 50% amplitude bursts, preferably 40 to 45% amplitude bursts, most preferably 45% amplitude bursts. In one embodiment, the water-in-oil core emulsion is subjected to sonication for 2 to 12 bursts, preferably 3 to 12 bursts, preferably 4 to 12 bursts, preferably 5 to 12 bursts, preferably 6 to 12 bursts, preferably 7 to 12 bursts, preferably 8 to 12 bursts, preferably 9 to 12 bursts, preferably 10 to 12 bursts, preferably 11 to 12 bursts, most preferably 12 bursts. In a preferred embodiment, the water-in-oil core emulsion is subjected to sonication at 45% amplitude bursts of 45 sec for 12 bursts. In one embodiment, the water-in-oil core emulsion is held in an ice bath between each burst of sonication for 0.5 to 6 min, preferably 1 to 5.5 min, preferably 1 to 5 min, preferably 1 to 4.5 min, preferably 1 to 4 minutes, preferably 1 to 3.5 min, preferably 1 to 3 min, preferably 1 to 2.5 min, preferably 1 to 2 min, preferably 1 to 1.5 min, most preferably 1 min. In a preferred embodiment, the water-in-oil core emulsion is held in an ice bath between each burst of sonication for 1 minute. The water-in-oil core emulsion thus prepared is a stable mixture of two immiscible liquids:water (the first aqueous phase) and oil (the oil phase).

At step 56, the method 50 comprises dispersing the water-in-oil core emulsion into a second aqueous phase. The second aqueous phase comprises a second surfactant and a co-surfactant. In some embodiments, the second surfactant may be, but is not limited to, an alkylglucoside, an alkylmaltoside, an alkylthioglucoside, a lauryl macrogolglyceride, a polyoxyethylene alkyl ether, a polyoxyethylene alkylphenol, a polyethylene glycol fatty acid ester, a polyethylene glycol glycerol fatty acid ester, a polyoxyethylene sorbitan fatty acid ester, a polymyethylene-polyoxypropylene block copolymer, a polyglycerol fatty acid ester, a polyoxyethylene glyceride, a polyoxyethylene sterol and any derivatives/analogues thereof, a polyoxyethylene vegetable oil, a polyoxyethylene hydrogenated vegetable oil, a reaction mixture of polyols with fatty acids, glycerides, vegetable oils, hydrogenated vegetable oils, and sterols, a sugar ester, a sugar ether, a sucroglyceride, a polyethoxylated fat-soluble vitamin or a derivative thereof, and mixtures thereof. In a preferred embodiment, the second surfactant is selected from the group consisting of a glyceryl citrate, a glyceryl lactate, a glyceryl linoleate, and a glyceryl oleate.

In some embodiments, the co-surfactant may be, but is not limited to, a mono-alcohol, a poly-alcohol, an organic acid, a salt of an organic acid, an amine, a polyethylene glycol, an ethoxylated solvent and combinations thereof. In a preferred embodiment, the co-surfactant is at least one selected from the group consisting of a polysorbate, a polyglycerol alkyl ether, a glucosyl dialkyl ether, a sorbitan ester, and a polyoxyethylene alkyl ether. In another preferred embodiment, the co-surfactant is a polysorbate.

In some embodiments, the co-surfactant is present in an amount of 1 to 20 wt. %, preferably 2 to 19 wt. %, preferably 3 to 18 wt. %, preferably 4 to 17 wt. %, preferably 5 to 16 wt. %, preferably 6 to 15 wt. %, preferably 7 to 14 wt. %, preferably 7 to 13 wt. %, preferably 7 to 12 wt. %, preferably 7 to 11 wt. %, preferably 7 to 10 wt. %, preferably 7 to 9 wt. %, preferably 7 to 8 wt. %, most preferably 7 wt. %. In some embodiments, the non-ionic surfactant is present in an amount of 1 to 20 wt. %, preferably 2 to 19 wt. %, preferably 3 to 18 wt. %, preferably 4 to 17 wt. %, preferably 5 to 16 wt. %, preferably 6 to 15 wt. %, preferably 7 to 14 wt. %, preferably 7 to 13 wt. %, preferably 7 to 12 wt. %, preferably 7 to 11 wt. %, preferably 7 to 10 wt. %, preferably 7 to 9 wt. %, preferably 7 to 8 wt. %, most preferably 7 wt. %.

The second aqueous phase is prepared by mixing the second surfactant and the co-surfactant to achieve a ratio of the second surfactant to the co-surfactant of 1:4 to 4:1, preferably 1:3 to 3:1, preferably 1:2 to 2:1, most preferably 1:1. In a preferred embodiment, the second surfactant and the co-surfactant are present in a ratio of 1:1 second surfactant to co-surfactant.

The water-in-oil core emulsion is dispersed into the second aqueous phase to form the multi-compartment nanoemulsion (W/O/W-NE). In one embodiment, the multi-compartment nanoemulsion is subjected to sonication at a power of 100 to 140 Watts (W) and a frequency of 10 to 30 kHz. In one embodiment, the multi-compartment nanoemulsion is subjected to sonication at a power of 105 to 135 W, preferably 110 to 130 W, preferably 115 to 125 W, preferably 120 to 125 W, most preferably 125 W. In one embodiment, the multi-compartment nanoemulsion is subjected to sonication at a frequency of 12 to 28 kHz, preferably 14 to 26 kHz, preferably 16 to 24 kHz, preferably 18 to 22 kHz, most preferably 20 kHz. In a preferred embodiment, the multi-compartment nanoemulsion is subjected to sonication at a power of 125 W and a frequency of 20 kHz for 15 to 60 sec at 20 to 65% amplitude bursts for 1 to 20 bursts. In one embodiment, the multi-compartment nanoemulsion is subjected to sonication for 20 to 55 sec, preferably 25 to 50 sec, preferably 30 to 45 sec, preferably 35 to 45 sec, preferably 40 to 45 sec, most preferably 45 sec. In one embodiment, the multi-compartment nanoemulsion is subjected to sonication at 25 to 60% amplitude bursts, preferably 30 to 60% amplitude bursts, preferably 35 to 60% amplitude bursts, preferably 40 to 60% amplitude bursts, preferably 45 to 60% amplitude bursts, preferably 50 to 60% amplitude bursts, preferably 55 to 60% amplitude bursts, most preferably 60% amplitude bursts. In one embodiment, the multi-compartment nanoemulsion is subjected to sonication for 2 to 12 bursts, preferably 3 to 12 bursts, preferably 4 to 12 bursts, preferably 5 to 12 bursts, preferably 6 to 12 bursts, preferably 7 to 12 bursts, preferably 8 to 12 bursts, preferably 9 to 12 bursts, preferably 10 to 12 bursts, preferably 11 to 12 bursts, most preferably 12 bursts. In a preferred embodiment, the multi-compartment nanoemulsion is subjected to sonication at 60% amplitude bursts of 45 sec for 12 bursts. In one embodiment, the multi-compartment nanoemulsion is held in an ice bath between each burst of sonication for 0.5 to 6 min, preferably 1 to 5.5 min, preferably 1 to 5 min, preferably 1 to 4.5 min, preferably 1 to 4 minutes, preferably 1 to 3.5 min, preferably 1 to 3 min, preferably 1 to 2.5 min, preferably 1 to 2 min, preferably 1 to 1.5 min, most preferably 1 min. In a preferred embodiment, the multi-compartment nanoemulsion is held in an ice bath between each burst of sonication for 1 minute.

The water-in-oil core emulsion has an average droplet size of less than 35 d·nm, preferably less than 30 d·nm, preferably less than 25 d·nm, preferably less than 20 d·nm, most preferably 18.5±0.44 d·nm. The multi-compartment nanoemulsion has an average droplet size of less than 100 d·nm, preferably 50 to 95 d·nm, preferably 55 to 90 d·nm, preferably 60 to 85 d·nm, preferably 65 to 80 d·nm, preferably 70 to 80 d·nm, most preferably 75 to 80 d·nm. In another preferred embodiment, the average droplet size is 77.70±0.55 d·nm.

The polydispersity index (PDI) measures the distribution of particle sizes within the composition. Polydispersity indexes range from 0 to 1, where very close or equal to 1 indicates extremely broad size distribution (e.g., a polydisperse system). If the PDI is closer to zero, this means only one size of particle is present, which denotes a monodisperse system. PDI is an important factor in the design of intranasal drug delivery systems because it indicates the size distribution of the droplets, with a smaller PDI indicating a more uniform drug absorption through the nasal mucosa. The water-in-oil core emulsion has a PDI of 0.03 to 0.06, preferably 0.035 to 0.06, preferably 0.040 to 0.06, preferably 0.045 to 0.06, preferably 0.05 to 0.06, preferably 0.055 to 0.06, most preferably 0.058. The multi-compartment nanoemulsion has a PDI of 0.01 to 0.30, preferably 0.02 to 0.29, preferably 0.03 to 0.28, preferably 0.04 to 0.27, preferably 0.05 to 0.26, preferably 0.06 to 0.25, preferably 0.07 to 0.24, preferably 0.08 to 0.23, preferably 0.09 to 0.22, preferably 0.10 to 0.21, preferably 0.11 to 0.20 preferably 0.12 to 0.19, preferably 0.13 to 0.18, preferably 0.14 to 0.17, preferably 0.15 to 0.16, most preferably 0.158. This value indicates that the droplets of both the water-in-oil core emulsion and the multi-compartment nanoemulsion are monodispersed, suggesting that the droplets are of substantially similar size which improves the stability of the multi-compartment nanoemulsion by minimizing coalescence (the merging of droplets), minimizing phase separation, and increasing drug adsorption and bioavailability upon intranasal administration.

At step 58, method 50 comprises administering the nanoemulsion to a nasal cavity of a subject in need thereof. Administering an effective amount of the pharmaceutical composition of the invention to a subject in need thereof is for the prevention, alleviation and/or treatment of epileptic seizures. In some embodiments, the subject can be an animal or a mammal. In a preferred embodiment, the subject is a human.

The multi-compartment nanoemulsion exhibits a favorable droplet size, dispersity, and stability, indicating its potential as an intranasal drug delivery system. This nanoemulsion holds promise for delivering medications directly and rapidly to the brain via olfactory neurons, offering a safe and efficient alternative to oral and intravenous drug administration to treat or prevent seizures or seizure-related disorders. These nanomedicine-based drug delivery systems have demonstrated great promise in the therapeutic management of brain disorders by effectively delivering drugs to the target of interest and bypassing the Blood-brain barrier (BBB), accessing the brain via the olfactory pathway, serving as a carrier for multiple drugs, and enhancing drug distribution within the brain. This integration of two antiseizure medications into a multi-compartment nanoemulsion facilitates their future use as an efficient drug delivery system to the brain.

### EXAMPLES

The following examples demonstrate an intranasal drug delivery system that is a multi-compartment nanoemulsion (W/O/W-NE) formulation loaded with levetiracetam and valproic acid. The examples are provided solely for illustration and are not to be construed as limitations of the present disclosure, as many variations thereof are possible without departing from the spirit and scope of the present disclosure.

### Example 1: Materials

IMWITOR® 600 (Polyglyceryl-3 Polyricinoleate), MIGLYOL® 812 N (Medium-Chain Triglyceride), and IMWITOR® 375 (Glyceryl Citrate/Lactate/Linoleate/Oleate), purchased from IoI oleochemical pharma, (Putrajaya, Malaysia). TWEEN® 80 (Polysorbate 80), Sigma Aldrich, USA. Valproic Acid obtained from Supelco, Germany. Levetiracetam procured from Sigma-Aldrich, USA. Water was chosen as a vehicle for levetiracetam and valproic acid.

### Example 2: Preparation of Water-In-Oil-Nanoemulsion (W/O-NE)

Valproic acid and levetiracetam was accurately weighed and dissolved in sterile distilled water with a pH of 7 to yield a concentration of 50 mg/ml and 133 mg/ml, respectively. In an Eppendorf tube, 400 microliters (μL) of Miglyol® was introduced first, followed by 300 μL of surfactant (IMWITOR® 600), and finally 300 μL of water.

The sample was subjected to sonication using an ultrasonic processor (125 w 20 kHz) for 45 seconds at 45% amplitude 12 bursts (Q125 Sonicator®, Qsonica, USA). A probe tip of the sonicator with 0.125″ diameter was used then the tip was submerged to a depth of 25 millimeter (mm) in the Eppendorf tube. During sonication the sample was kept in an ice bath between each burst for 1 minute to ensure nanoemulsion not heat up. The water-in-oil core emulsion (W/O-NE) was created from a naturally occurring pharmaceutical, 40% coconut oil (Miglyol 812), 30% Imwitor 600 (polyglyceryl-3 polyricinoleate), and 30% purified distilled water. The first aqueous phase of the W/O-NE is made of water (to solubilize water-soluble drugs) and stabilized by Imwitor 600 at the interface, while the outer layer is composed of Miglyol 812. W/O-NE was then utilized to prepare the W/O/W-NE.

### Example 3: Determination of Droplet Size and PDI for W/O NE

To avoid a multi-scattering influence the sample diluted, 10 μL of the formulation was added to 990 μL Migloyl® (ratio 1:10 v/v), then vortexing the sample at 2500 rpm until formulation properly mixed with the oil. Then, the sample transferred to cuvette to quantify the size of the nanoemulsion droplets and the polydispersity indexes (PDIs) by Zetasizer (Malvern, Worcestershire, UK). Assessments were done in triplicate. The droplet size and dispersity of drug-loaded W/O NEs was found to be 18.5±0.44 d·nm with a polydispersity index (PDI) of 0.058, indicating that W/O NEs nanodroplets are monodispersed. Monodispersity is crucial factor for to study pharmacokinetics and pharmacodynamics during pharmaceutical applications. Having a W/O-NE droplet size below 20 d·nm facilitates the development of the W/G/W-NE.

### Example 4: Preparation of W/O/W-NEs

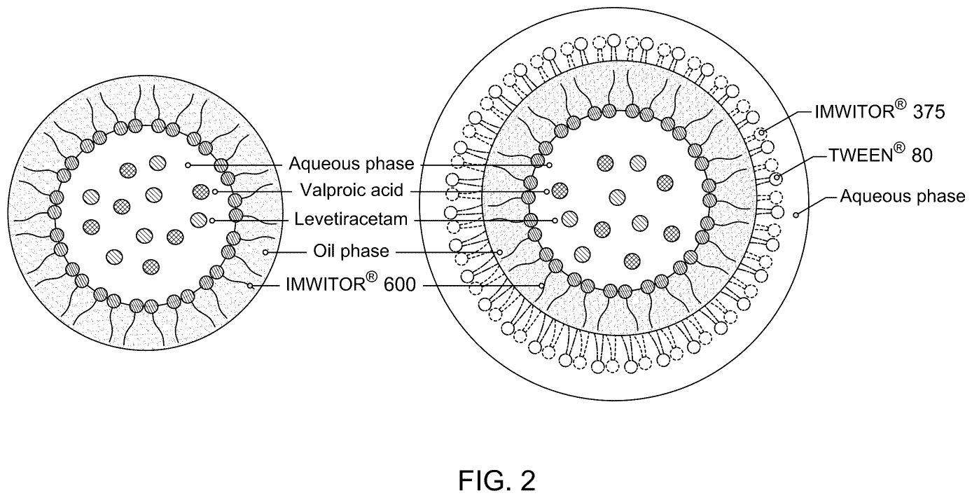

In the final step, the obtained water-in-oil core emulsion (W/O-NE) was further encapsulated in a second aqueous phase to yield a W/G/W-NE. The W/G/W-NE was stabilized with a second surfactant and cosurfactant, 7% Imwitor of 375 and 7% of Tween 80, respectively and a final concentration of 10% Medium-Chain Triglyceride, Miglyol 812 (FIG. 2). The aim of this is to create another hydrophobic core within the multi-compartment W/G/W-NE for loading lipophilic drugs and transforming the outer layer into a hydrophilic surface for better bio-interactions. Again, the sample was subjected to sonication using an ultrasonic processor for 45 seconds at 60% amplitude 12 bursts. During sonication, the sample was kept in an ice bath between each burst for 1 minute to ensure nanoemulsion not heat up.

### Example 5: Determination of Droplet Size and PDI for W/O/W-NE

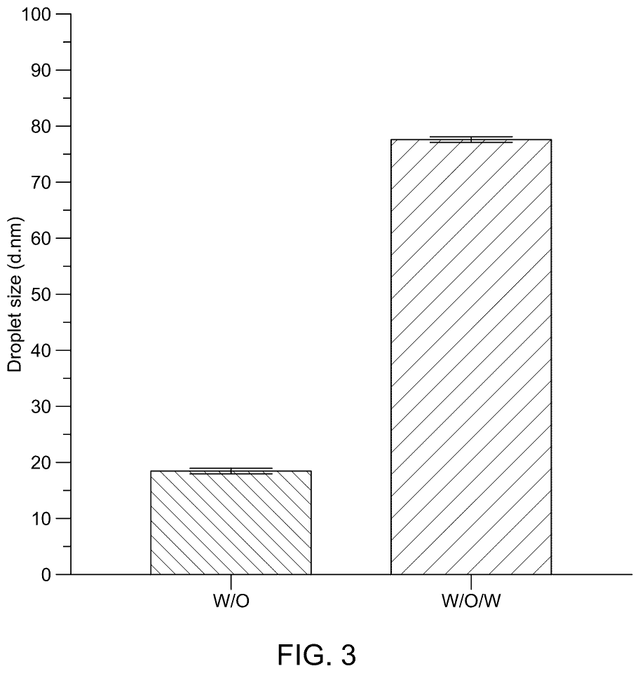

The sample diluted, 10 μL of the formulation was added to 990 μL water (ratio 1:10 v/v) then undergo size and PDI analysis in Zetasizer. The W/O/W-NEs demonstrated an average droplet size of 77.70±0.55 d·nm with a PDI of 0.158. Achieving droplet sizes <100 d·nm is an important factor for intranasal drug delivery and bypassing the blood brain barrier (BBB) (FIG. 3).

### Example 6: Determination of Stability for W/O/W-NE

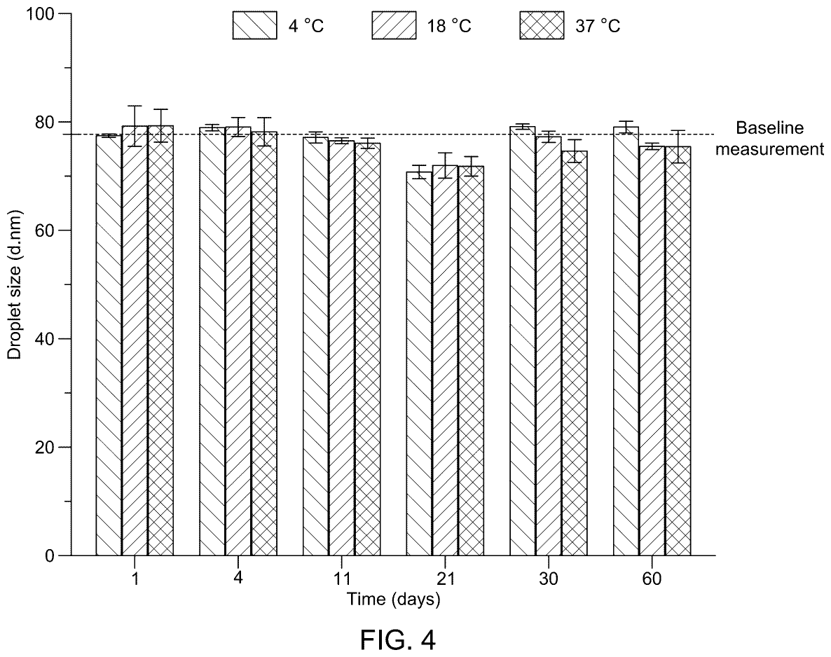

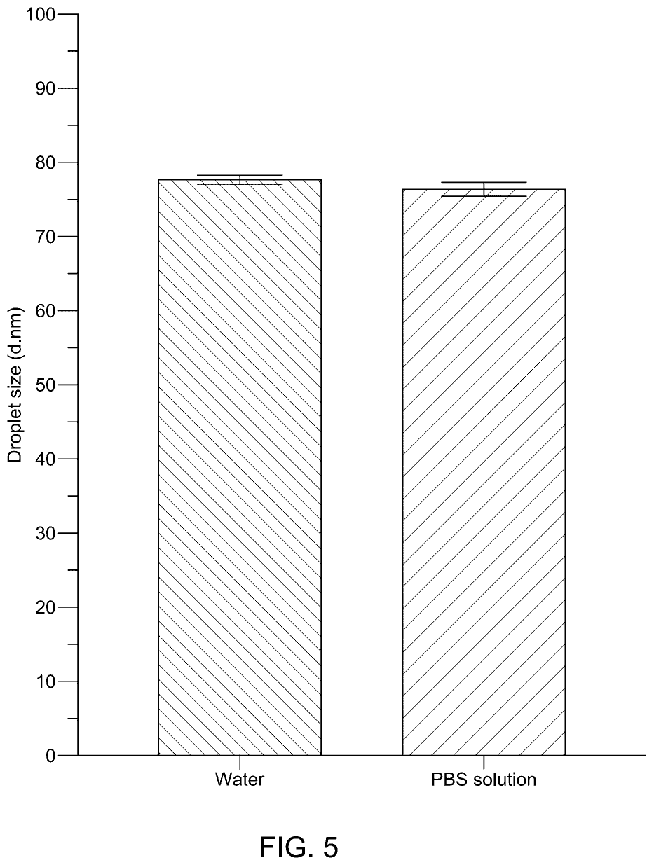

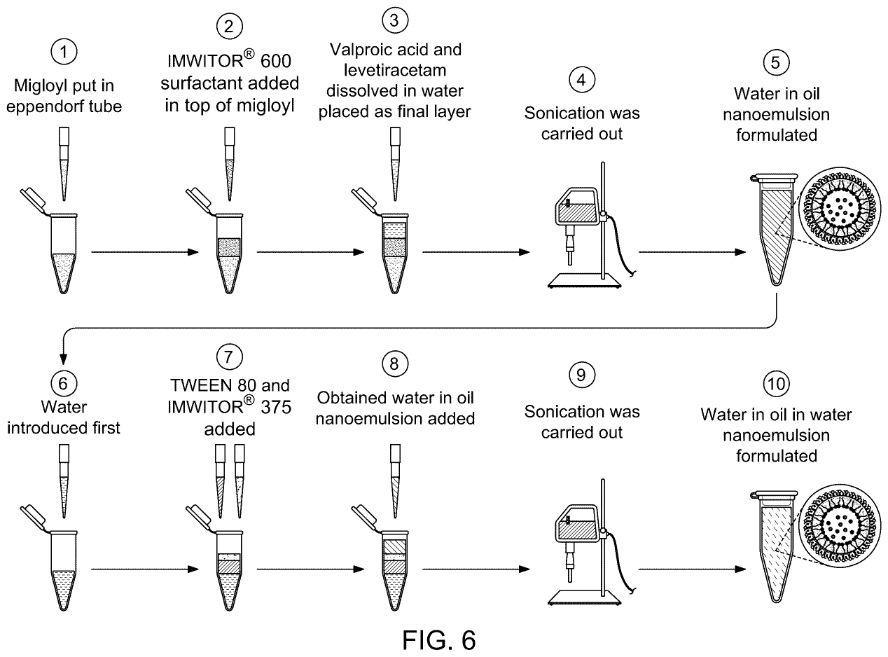

The W/O-NE and W/O/W-NE formulations retained their physiochemical characteristics, did not show any separation or creaming, and were stable for 60 days at low temperature (4° C.), room temperature and body temperature (37° C.) (FIG. 4). W/O/W-NE has also shown stability against high salt concentration solution and phosphate buffer saline (PBS), which mimics physiological conditions (FIG. 5). The formulated multi-compartment nanoemulsion exhibits an optimal size, dispersity, and stability, indicating its potential as a drug delivery system for anti-seizure drugs. This formulation thus aids in delivering medications directly and rapidly to the brain via olfactory neurons, offering a safe and efficient alternative to intravenous administration to treat status epilepticus. FIG. 6 depicts the W/O/W-NEs preparation process.

### Example 7: In Vivo Studies of W/O/W-NE Delivered Intranasally

The local mucociliary toxicity effect of the W/O/W-NEs was investigated on sheep mucosa in comparison to pH 6.5 PBL, as a negative control, and isopropyl alcohol, as positive control. The head of a 1-year-old sheep was collected immediately after sacrificing from a local slaughterhouse (Jeddah, Saudi Arabia). Within 15 minutes of sacrifice, the nasal cavity was exposed, and the mucus membrane was removed, washed, and cut into three symmetrical pieces of similar thicknesses (about 2 mm). The first piece was submerged in the W/O/W-NEs for one hour, and then rinsed with PBS (pH of 6.5). The second and third pieces were submerged in isopropyl alcohol (a strong mucociliary toxin) and PBS (pH 6.5) respectively. Samples from the pieces were immersed in 10% neutral buffer formalin (NBF) fixation solution for 48 hours. The nasal mucosal samples were embedded in paraffin to obtain paraffin blocks, sectioned by microtome into 4 μm slices. Nasal mucosa samples were then mounted on glass slides and stained with a combination of hematoxylin and eosin (H&E) dye. The slides were examined under an optical microscope at 40× (Olympus, Japan) to detect cilia damage and morphological changes in the nasal mucosa.

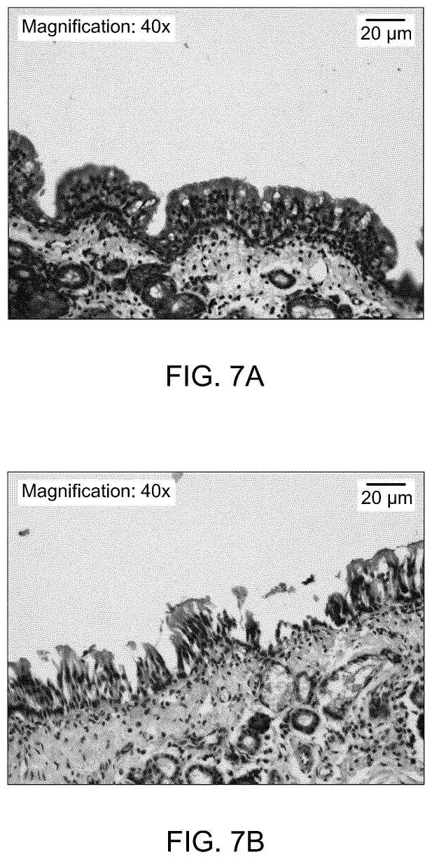

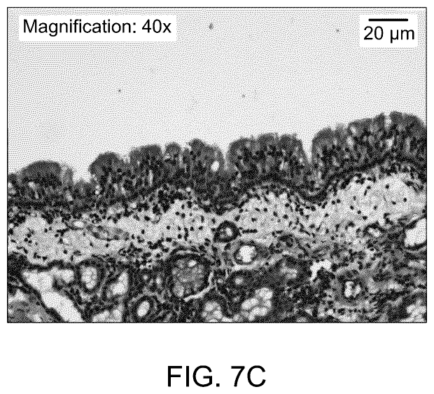

As shown in FIG. 7A to FIG. 7C, nasal mucosa in the negative control revealed intact stratified squamous epithelium and normal connective tissue while nasal mucosa exposed to isopropyl alcohol showed sloughing of epithelial layer. Applying a solution comprising the W/O/W-NEs to the nasal mucosa showed normal structure of epithelial layer with no signs of erosion and inflammation.

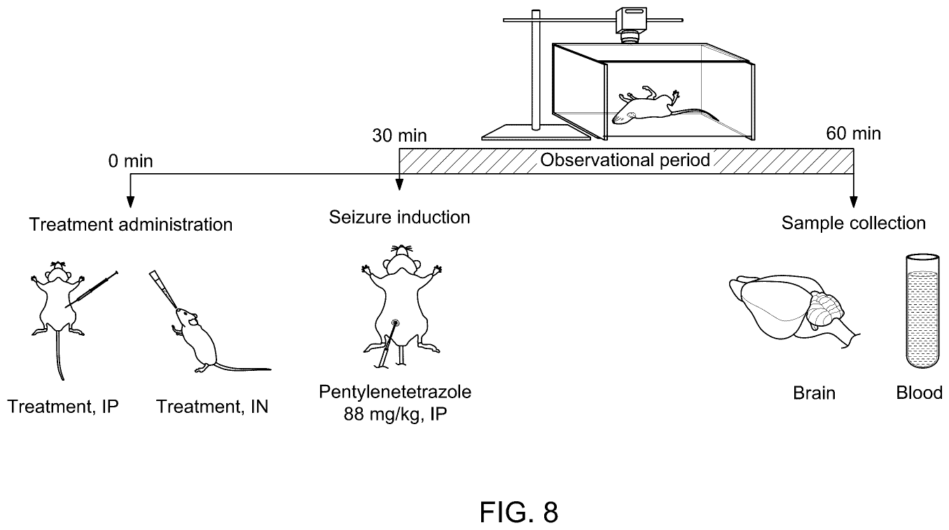

The effect of different routes and formulation of valproate (VPA) and/or levetiracetam (LEV) on a mice model of PTZ-induced seizure was further investigated by measuring the latency of seizure, number of generalized tonic-clonic seizures (GTCS), duration of GTCS, and severity of seizures scored according to modified Racine's scale (Table 1). The experimental procedure is depicted in FIG. 8.

TABLE 1
Behavioral seizure assessment according to a modified Racine scale

| Seizure Stage | Behavioral Expression | Score |
|---|---|---|
| 0 | No changes in behavior | |
| 1 | Myoclonic jerks with sudden and repetitive movement | 1 |
| | of the head and neck with or without tail stiffening | |
| 2 | Atypical (unilateral or incomplete) clonic seizure | 2 |
| 3 | Clonic seizure with forelimb clonus and rearing | 3 |
| 4 | Tonic-clonic seizure with an initial wild run | 4 |
| | and subsequent loss of righting reflex | |
| 5 | Tonic-clonic seizure with full extension of | 5 |
| | fore- and hind-limbs | |

Swiss mice (SWR/J) male mice weighing (30±2 g) were obtained and housed from animal house unit at King Fahd Medical Research Center, King Abdulaziz University, Jeddah, Saudi Arabia. The mice were kept in cages of 5 mice, temperature-controlled environment, a standard 12/12-hour light/dark cycle, with free access to water and food. All experiments were performed according to the guidelines of the biomedical ethics research committee at King Abdulaziz University and followed the rules and regulations of the Animal Care and Use Committee at the KFMRC which comply with guidelines of “System of ethics of research on living creatures” prepared by King Abdulaziz City for Science and Technology and were approved by the Royal Decree No. M/59 dated 24 Aug. 2010. This experiment used pentylenetetrazol (PTZ) as an acute seizure induction model, with an induction dose of 88 mg/kg. PTZ. The drug formulation was administered 30 minutes before seizure induction, and it was instilled into the nostrils with a micropipette (P100) with a volume of not more than 100 μl based on mouse weight.

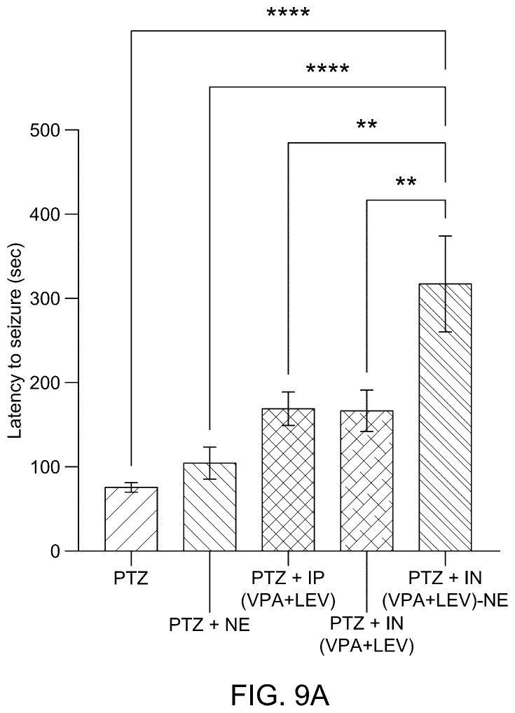

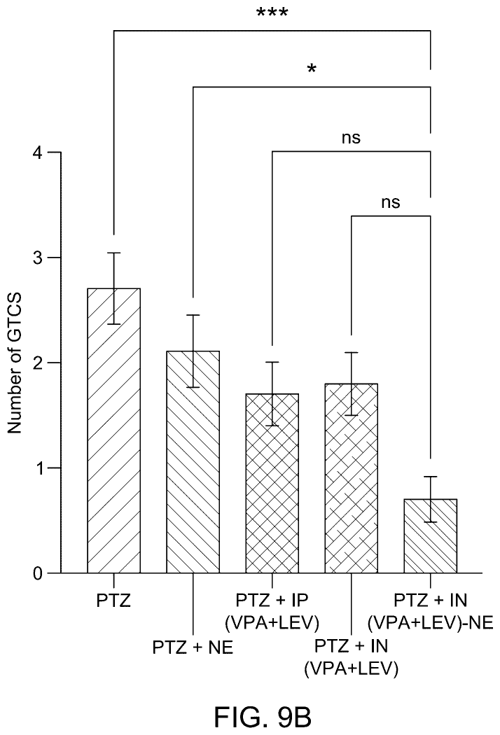

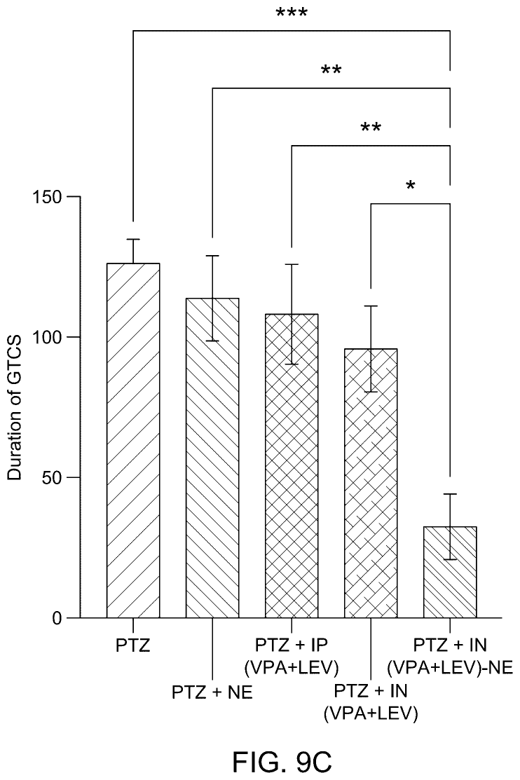

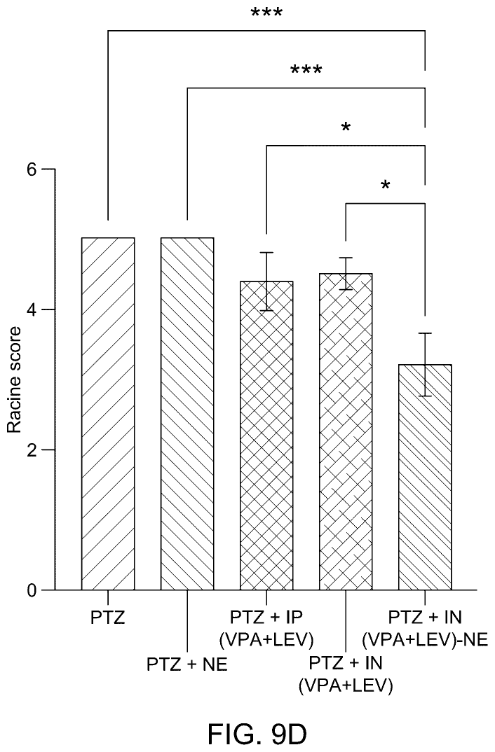

Mice were randomly distributed into five groups (n=10 in each group). Group I (PTZ) intraperitoneal PTZ (88 mg/kg) and intranasal (IN) saline, Group II (PTZ+NE) received PTZ and IN drug-free W/O/W-NEs, Group III (PTZ+IP (VPA+LEV)) received PTZ and an intraperitoneal solution of LEV (10 mg/kg) and VPA (4 mg/kg), Group IV (PTZ+IN (VPA+LEV)) received PTZ and an IN solution of LEV (10 mg/kg) and VPA (4 mg/kg), and Group V (PTZ+IN (VPA+LEV)-NE) received the IN nanoemulsion (W/O/W-NE) of LEV (10 mg/kg) and VPA (4 mg/kg). Immediately after PTZ injection, mice were placed individually in cages and continuously observed for 30 minutes, with an individual record for each animal using a digital camera placed on top of the observation box. Over the observation period, animals were assessed for latency of seizure, which is the time between PTZ administration and seizure onset, measured in seconds, the number of GTCS with seizures graded as a stage 4 and 5 being counted, duration of GTCS, which is the time (in seconds) it takes from the commencement of the seizure until the mice recovers, and the severity of seizures as scored according to the modified Racine's scale, as shown in Table 1. Data revealed that the W/O/W-NEs formulation of VPA and LEV, administered intranasally, significantly reduced the latency to seizure compared to PTZ group (p<0.0001); PTZ+NE (p<0.0001); PTZ, as shown in FIG. 9A to FIG. 9D. Data in FIG. 9A through FIG. 9D is presented as mean±SEM. One-way ANOVA followed by Tukey's multiple comparisons test was used, 5 * p<0.1, ** p<0.01, **** p<0.0001.

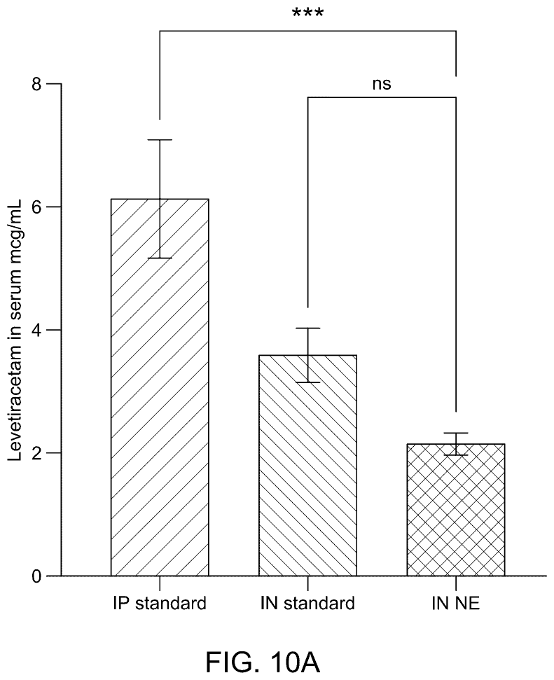

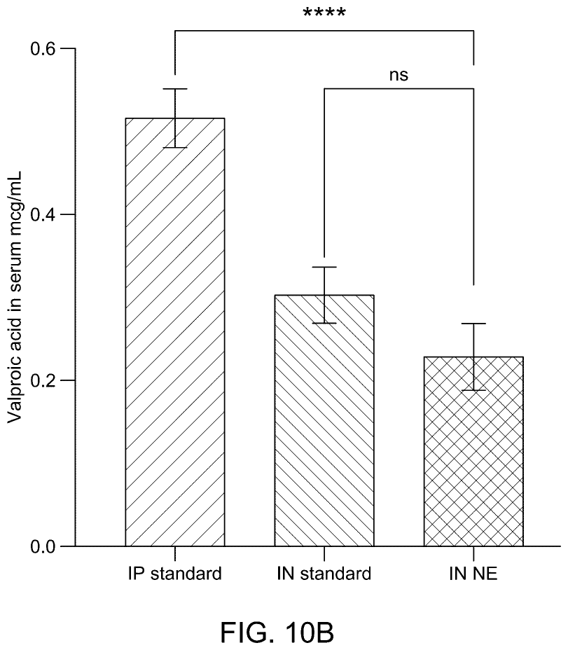

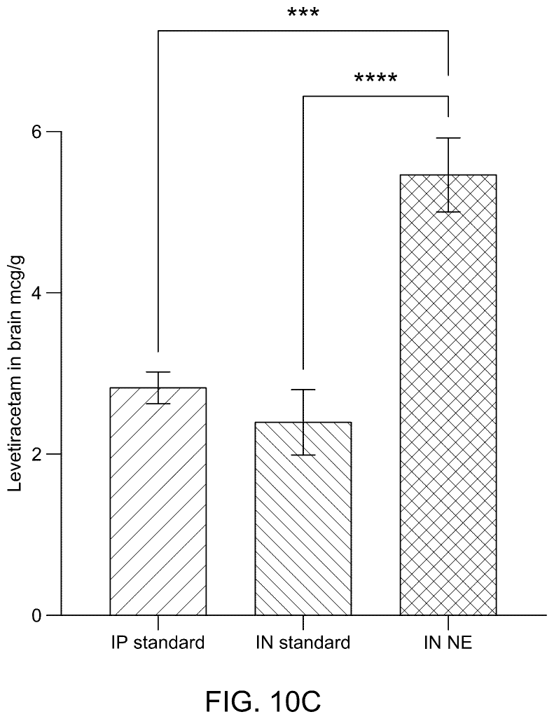

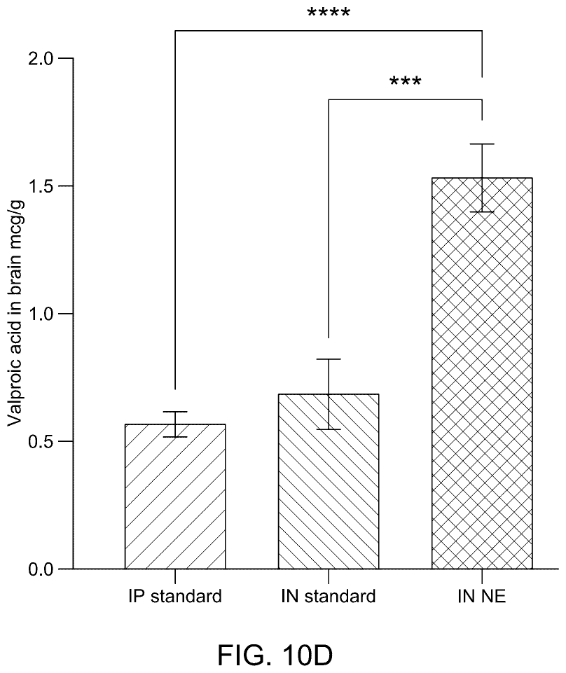

Additionally, the concentration VPA and LEV in the brain after administration to the mice was measured using Liquid Chromatography-Mass Spectrometry (LC-MS). Groups III and IV (n=7) were used as the IP and IN standards, respectively. Both the plasma concentration and brain concentration of VPA and LEV were measured 60 minutes after administration (FIG. 10A to FIG. 10D). The results of these measurements are shown in Table 2 and Table 3. The intranasally administered W/O/W-NE showed higher concentrations in the brain for both VPA and LEV compared to the IP and IN standards.

TABLE 2
Concentration of VPA in plasma and brain.

| | | Plasma concentration | Brain concentration | Brain:plasma |
|---|---|---|---|---|
| | | after 60 min (μg/mL) | after 60 min (μg/mL) | ratio after |
| Preparation | Route | mean ± SEM | mean ± SEM | 60 min |
| IP standard | Intraperitoneal | 0.5157 ± 0.034 | 0.570 ± 0.045 | 1.106 |
| IN standard | Intranasal | 0.3033 ± 0.03 | 0.686 ± 0.136 | 2.261 |
| IN-NE | Intranasal | 0.2284 ± 0.039 | 1.534 ± 0.131 | 6.728 |

TABLE 3
Concentration of LEV in plasma and brain.

| | | Plasma concentration | Brain concentration | Brain:plasma |
|---|---|---|---|---|
| | | after 60 min (μg/mL) | after 60 min (μg/mL) | ratio after |
| Preparation | Route | mean ± SEM | mean ± SEM | 60 min |
| IP standard | Intraperitoneal | 6.133 ± 0.958 | 2.827 ± 0.191 | 0.460 |
| IN standard | Intranasal | 3.609 ± 0.421 | 2.400 ± 0.402 | 0.665 |
| IN-NE | Intranasal | 2.168 ± 0.1578 | 5.468 ± 0.455 | 2.522 |

In the present disclosure a multi-compartment nanoemulsion capable of simultaneously encapsulating levetiracetam and valproic acid is described, marking a significant advancement for intranasal applications. The nanoemulsion formulation, stabilized with a mix of a surfactant and a co-surfactant, demonstrates impressive long-term physical stability and retains its nanoparticle size under various conditions. This innovative approach enhances the potential for effective drug delivery through multiple routes, contributing to improved therapeutic outcomes for treatment and/or prevention of seizures.

Numerous modifications and variations of the present disclosure are possible in light of the above teachings. It is therefore to be understood that within the scope of the appended claims, the invention may be practiced otherwise than as specifically described herein.

1. A method of intranasal drug delivery, comprising:
administering a multi-compartment nanoemulsion to an oral cavity of a subject in need thereof,
wherein the multi-compartment nanoemulsion comprises a water-in-oil core emulsion having an oil phase encompassing a first aqueous phase, the oil phase comprising a first surfactant and a fatty acid and the first aqueous phase comprising levetiracetam and valproic acid dissolved therein,
wherein the multi-compartment nanoemulsion further comprises a second aqueous phase encompassing the water-in-oil core emulsion, the second aqueous phase comprising a second surfactant and a co-surfactant in a ratio of the second surfactant to the co-surfactant of 1:4 to 4:1,
wherein the water-in-oil core emulsion has an average droplet size of 20 d·nm or less and the multi-compartment nanoemulsion has an average droplet size of less than 100 d·nm,
wherein the first surfactant is polyglyceryl-3 polyricinoleate,
wherein the co-surfactant is a polysorbate, and
wherein the multi-compartment nanoemulsion has a polydispersity index (PDI) of 0.1 to 0.2.

2. The method of claim 1, further comprising:
preparing the first aqueous phase by mixing levetiracetam and valproic acid in water;
forming the oil phase, then dispersing the first aqueous phase into the oil phase to obtain the water-in-oil core emulsion; and
dispersing the water-in-oil core emulsion into the second aqueous phase, then sonicating to obtain the multi-compartment nanoemulsion.

3. The method of claim 1, wherein the second surfactant is present in an amount of 1 to 10 wt. %.

4. The method of claim 1, wherein the co-surfactant is present in an amount of 1 to 10 wt. %.

5. The method of claim 1, wherein the multi-compartment nanoemulsion has an average droplet size of less than 85 d·nm.

6. The method of claim 1, wherein the fatty acid is at least one selected from the group consisting of a coconut oil, a palm kernel oil, a caproic acid, a caprylic acid, a capric acid, and a lauric acid.

7. The method of claim 1, wherein the second surfactant is present in an amount of 7 wt.

8. The method of claim 1, wherein the co-surfactant is present in an amount of 7 wt. %.

9. The method of claim 1, wherein the second surfactant is at least one selected from the group consisting of a glyceryl citrate, a glyceryl lactate, a glyceryl linoleate, and a glyceryl oleate.

10. The method of claim 2, wherein the sonicating comprises sonicating the aqueous suspension at 25 to 65% amplitude bursts for 15 to 60 seconds; and holding the aqueous suspension in an ice bath between each burst for 0.5 to 6 minutes.

11. The method of claim 2, wherein the forming of the oil phase comprises adding the fatty acid to the first aqueous phase, then adding the first surfactant to the fatty acid in a ratio of fatty acid to first surfactant of 1:1 to 3:1.

12. The method of claim 2, wherein the sonicating comprises sonicating the aqueous suspension at 45% amplitude bursts for 45 seconds; and holding the aqueous suspension in an ice bath between each burst for 1 minute.

13. The method of claim 1, wherein the multi-compartment nanoemulsion has a PDI of 0.158.

14. The method of claim 1, wherein the multi-compartment nanoemulsion has an average droplet size of 77.7±0.55 d·nm.

15. The method of claim 1, wherein the water-in-oil core emulsion has a PDI of 0.03 to 0.06.

16. The method of claim 1, wherein the water-in-oil core emulsion has a PDI of 0.058.

17. The method of claim 1, wherein the water-in-oil core emulsion has an average droplet size of 18.5±0.44 d·nm.

18. The method of claim 1, wherein the fatty acid is coconut oil.

19. The method of claim 1, wherein the levetiracetam is present in the first aqueous phase in a concentration of 100 to 150 mg/mL.

20. The method of claim 1, wherein the valproic acid is present in the first aqueous phase in a concentration of 30 to 70 mg/mL.
